(19) United States
(12) Patent Application Publication
(10) Pub. No.: US 2004/0064033 A1
(43) Pub. Date: Apr. 1, 2004

(54) System and method for reconstructing k-space data

(76) Inventors: Brian Dale, Euclid, OH (US); Jeffrey L. Duerk, Avon Lake, OH (US)

Correspondence Address: CALFEE HALTER & GRISWOLD, LLP, 800 SUPERIOR AVENUE, SUITE 1400, CLEVELAND, OH, 44114, US

(21) Appl. No.: 10/435,954

(22) Filed: May 12, 2003

Related U.S. Application Data
(60) Provisional application No. 60/380,758, filed on May 14, 2002.

(51) Int. Cl. A61B 5/05

(52) U.S. Cl. 600/410

(57) ABSTRACT

Computer implemented methods that reduce artifacts in images reconstructed from MRI data acquired during planned radial acquisition trajectories are provided. One example method includes obtaining a planned radial trajectory along which MRI data should be acquired, predicting trajectories that may actually be taken during MRI data acquisition, and measuring an actual trajectory taken during MRI data acquisition. The method also includes acquiring an MRI data, where it is attempted to acquire the MRI data in accordance with the planned radial trajectory, and selectively reconstructing the MRI data into an image using one or more of, the planned radial trajectory, the measured trajectory, and the predicted trajectories based, at least in part, on an artifact level in a reconstructed image and discrepancies between planned trajectories and actual trajectories.

### CROSS REFERENCE TO RELATED APPLICATIONS

This application claims priority to U.S. Provisional Application No. 60/380,758 titled “The Use of Measured K-Space Trajectory for Reconstruction of Radial MRI Data”, filed May 14, 2002, which is incorporated herein by reference.

### TECHNICAL FIELD

This application relates to the image processing arts and more particularly to improving magnetic resonance imaging (MRI) system images.

### BACKGROUND

Magnetic resonance imaging systems acquire diagnostic images without relying on ionizing radiation. Instead, MRI employs strong, static magnetic fields, radio-frequency (RF) pulses of energy, and time varying magnetic field gradient waveforms. MRI is a non-invasive procedure that employs nuclear magnetization and radio waves for producing internal pictures of a subject. Two or three-dimensional diagnostic image data is acquired for respective “slices” of a subject area. These data slices typically provide structural detail having, for example, a resolution of one millimeter or better.

Programmed steps for collecting data, which is used to generate the slices of the diagnostic image, are known as a magnetic resonance (MR) image pulse sequence. The MR image pulse sequence includes generating magnetic field gradient waveforms applied along up to three axes, and one or more RF pulses of energy. The set of gradient waveforms and RF pulses facilitate collecting data for reconstructing the image slices.

Data is acquired during MR device excitation(s). The excitation(s) can be designed to follow various trajectories including radial trajectories. These trajectories, governed by the magnetic field gradient waveforms, facilitate acquiring data in K space, a concept known to those skilled in the art. Preferably, there are no timing problems or eddy currents experienced during image acquisition. However, such events do occur, leading, in some cases, to the introduction of artifacts into a reconstructed MR image. These artifacts may degrade the quality of the MR image. The artifacts typically occur due to differences in the desired and generated gradient waveforms, and resulting differences between the desired k-space trajectory sample data locations and those from which data is actually obtained.

Measured trajectories have been employed in spiral trajectories and rectilinear echo-planar imaging (EPI). However, measured trajectories have not conventionally been employed in radial k-space data acquisition. Factors including, but not limited to, timing errors, and uncompensated eddy currents can lead to deviations from a planned and/or designed k-space data acquisition trajectory. If the deviations are severe enough, the planned radial trajectory designed to acquired radial data may end up not being a radial trajectory and thus not acquiring radial data. These deviations can produce image artifacts like streaks. Radial acquisition techniques use gradient waveforms similar to standard Fourier imaging waveforms and thus image artifacts were considered unlikely in this type of acquisition technique. However, such image artifacts persisted, even in radial k-space data acquisition.

### SUMMARY

The following presents a simplified summary of methods, systems, Application Programming Interfaces (APIs), and computer readable media for improving MRI images by reconstructing images from radial k-space data using planned, actual/measured, and/or predicted trajectories, to facilitate providing a basic understanding of these items. This summary is not an extensive overview and is not intended to identify key or critical elements of the methods, systems, computer readable media, and so on or to delineate the scope of these items. This summary provides a conceptual introduction in a simplified form as a prelude to the more detailed description that is presented later.

This application describes systems and methods in which trajectory measurement and/or prediction techniques are applied to radial k-space data acquisition techniques. Flexible radial sequences are developed using, for example, pulse-sequence development tools. A trajectory measurement technique is employed to measure the trajectory and/or a prediction technique is employed to predict a trajectory. During data acquisition, a variety of parameters associated with the actual measured trajectory can be measured. After acquisition, the measured trajectory can be plotted and compared to a designed trajectory to facilitate identifying discrepancies between a designed and an actual trajectory. If discrepancies are found, image data acquired during the measured trajectory is reconstructed using measured and/or predicted trajectory data instead of and/or in association with planned and/or designed trajectory data.

This application describes a computer implemented method for reconstructing an image from an MRI data. In one example, a computer implemented method includes obtaining a planned radial trajectory, acquiring an MRI data, where it is attempted to acquire the MRI data in accordance with the planned trajectory, measuring the actual trajectory that occurs during data acquisition, and reconstructing an image from the acquired MRI data and the actual trajectory. In one example, the method includes reconstructing an image from the acquired data using a predicted radial trajectory. In yet another example, the method includes selectively reconstructing an image from the acquired data using one or more of a planned trajectory, a measured trajectory, and a predicted trajectory.

The application also describes a system for producing an MRI image. One example system includes a magnetic resonance imager for acquiring an MRI data and an image reconstructor for reconstructing an image from the MRI data. The image reconstructor can use data including, but not limited to, a measured trajectory, a planned trajectory, and a predicted trajectory to reconstruct the image.

The application also describes an API for execution by a computer component in conjunction with an application program that reconstructs an image from MRI data. One example API includes a first interface for passing an image data between, for example, programmers, processes, and an image reconstructor. An example API may also include a second interface for passing a planned trajectory data between, for example, programmers, processes, and an image reconstructor. Similarly, an example API may also include a third interface for passing a measured trajectory data between, for example, programmers, processes, and an image reconstructor. The image reconstructor can reconstruct an image from an MRI data from one or more of an image data, a planned trajectory data, and a measured trajectory data.

The application also describes a system that facilitates reconstructing an image from MRI data that mitigates artifacts in the image. One example system includes an MRI data, a planned trajectory data, an acquired trajectory data, and a data store for storing the MRI data, the planned trajectory data, and the acquired trajectory data. The example system also includes a trajectory comparator for comparing the planned trajectory data with the acquired trajectory data to produce a trajectory comparison data. An example system can also include an image reconstructor for reconstructing an image from the MRI data and one or more of, the trajectory data, and the acquired trajectory data based, at least in part, on the trajectory comparison data.

Certain illustrative example methods, systems, APIs, and computer readable media are described herein in connection with the following description and the annexed drawings. These examples are indicative, however, of but a few of the various ways in which the principles of the methods, systems, APIs, and computer readable media may be employed and thus are intended to be inclusive of equivalents. Other advantages and novel features may become apparent from the following detailed description when considered in conjunction with the drawings.

### BRIEF DESCRIPTION OF THE DRAWINGS

FIG. 1 illustrates example k-space trajectory samples affected by timing issues.

FIG. 2 illustrates example k-space trajectory samples affected by timing and/or eddy current issues.

FIG. 3 illustrates example k-space trajectory samples that do not exhibit errors due to timing and/or eddy current issues.

FIG. 4 compares example images reconstructed using planned and measured trajectories.

FIG. 5 illustrates a plot of k-space peak locations employed in predicting a trajectory.

FIG. 6 illustrates an example MRI system.

FIG. 7 is a flow chart illustrating one example method for reconstructing an image from MRI data.

FIG. 8 is a flow chart illustrating another example method for reconstructing an image from MRI data.

FIG. 9 is a schematic block diagram of an example computing environment with which the systems, methods, APIs, and computer readable media described herein may interact.

FIG. 10 illustrates an example API employed in accordance with an aspect of the present invention.

FIG. 11 illustrates an example MRI system interacting with an image reconstructing system.

FIG. 12 illustrates an example MRI system that includes an image reconstructing system.

### DETAILED DESCRIPTION

Example methods, systems, APIs, and computer media are now described with reference to the drawings where like reference numerals are used to refer to like elements throughout. In the following description, for purposes of explanation, numerous specific details are set forth to explain the methods, systems, APIs, and computer readable media. It may be evident, however, that the methods, systems, APIs, and computer readable media can be practiced without these specific details. In other instances, well-known structures and devices are shown in block diagram form in order to simplify description.

As used in this application, the term “computer component” refers to a computer-related entity, either hardware, firmware, software, a combination thereof, or software in execution. For example, a computer component can be, but is not limited to being, a process running on a processor, a processor, an object, an executable, a thread of execution, a program, and a computer. By way of illustration, both an application running on a server and the server can be computer components. One or more computer components can reside within a process and/or thread of execution and a computer component can be localized on one computer and/or distributed between two or more computers.

An operable connection is one in which signals and/or actual communication flow and/or logical communication flow may be sent and/or received. Usually, an operable connection includes a physical interface, an electrical interface, and/or a data interface, but it is to be noted that an operable connection may consist of differing combinations of these or other types of connections sufficient to allow operable control. Computer components may, in some cases, be “operably connected” by an operable connection.

“Software”, as used herein, includes but is not limited to, one or more computer readable and/or executable instructions that cause a computer or other electronic device to perform functions, actions and/or behave in a desired manner. The instructions may be embodied in various forms like routines, algorithms, modules, methods, threads, and/or programs. Software may also be implemented in a variety of executable and/or loadable forms including, but not limited to, a stand-alone program, a function call (local and/or remote), a servelet, an applet, instructions stored in a memory, part of an operating system or browser, and the like. It is to be appreciated that the computer readable and/or executable instructions can be located in one computer component and/or distributed between two or more communicating, co-operating, and/or parallel processing computer components and thus can be loaded and/or executed in serial, parallel, massively parallel, and other manners.

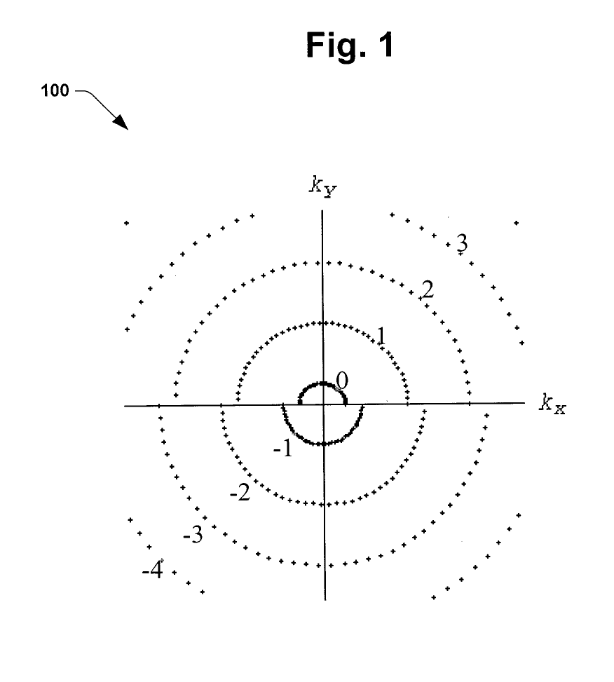

Referring initially to FIG. 1, k-space trajectory samples affected by timing issues are illustrated. FIG. 1 illustrates the central portion of a measured trajectory. The central portion is shown to facilitate visualizing data acquisition discrepancies that can lead to artifacts. The data displayed in FIG. 1 was acquired on a 1.5 T Siemens Sonata short bore magnet that includes a fast gradient system and active eddy current suppression. The data was acquired with a TR/TE=10 ms/5 ms and a technique known as FLASH acquisition. (TR=repetition time, TE=echo time). Trajectory measurement techniques (e.g., Duyn et al., J. Mag. Reson. 132: 150-153. 1998) can be employed to facilitate measuring the trajectory through the k-space.

Samples like those illustrated in FIG. 1 should yield a standard polar-coordinate grid presenting concentric, unbroken circles centered about the k-space origin if equidistant time sampling is employed during acquisitions and if acquisitions begin collecting data at the same point during gradient waveform application. Thus, if there were no discrepancies, the n and n-(n being an integer) semi-circles would meet and form circles. Also, if there were no discrepancies, the circles would be centered about the origin.

FIG. 1 illustrates trajectories with discrepancies. FIG. 1 shows circles broken into semi-circles, which is consistent with a timing error. These timing errors shift the k-space center in the read direction. In standard Fourier imaging, this yields a linear phase shift but does not change the magnitude of the image. In radial k-space acquisitions, the direction of phase shift varies as the readout direction moves through various angles. Changing the shift can cause artifacts. In one case, the artifact is perpendicular to an initial readout direction, which is illustrated as the direction of the transition from one set of semi-circles to another.

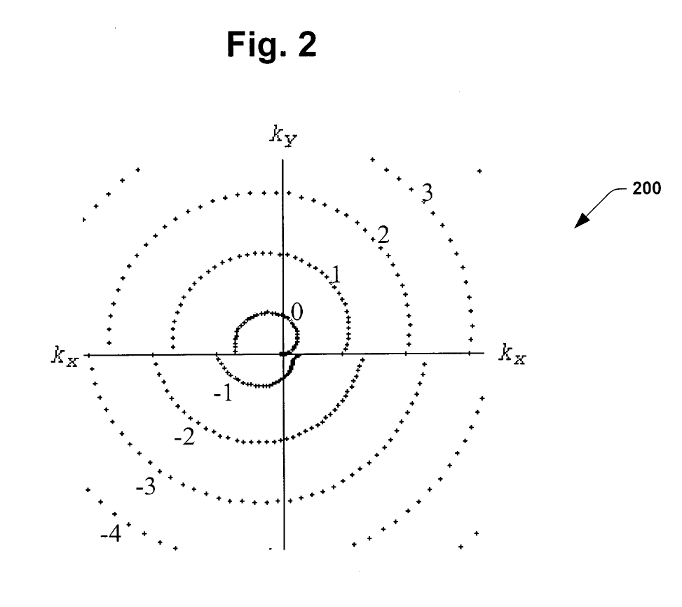

FIG. 2 illustrates the central portion of another measured trajectory. The data displayed in FIG. 2 was also acquired on a 1.5 T Siemens Sonata short bore magnet that included a fast gradient system and active eddy current suppression. The data was acquired with a TR/TE=100 ms/5 ms in a FLASH acquisition. Like FIG. 1, FIG. 2 clearly illustrates a discrepancy. In addition to the trajectory being broken from circles into semi-circles, the trajectory has also been distorted, which can lead to even more severe artifacts in an image reconstructed from the data.

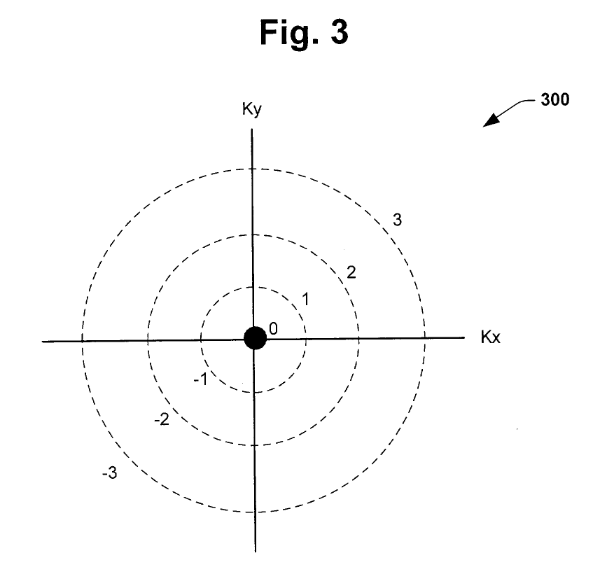

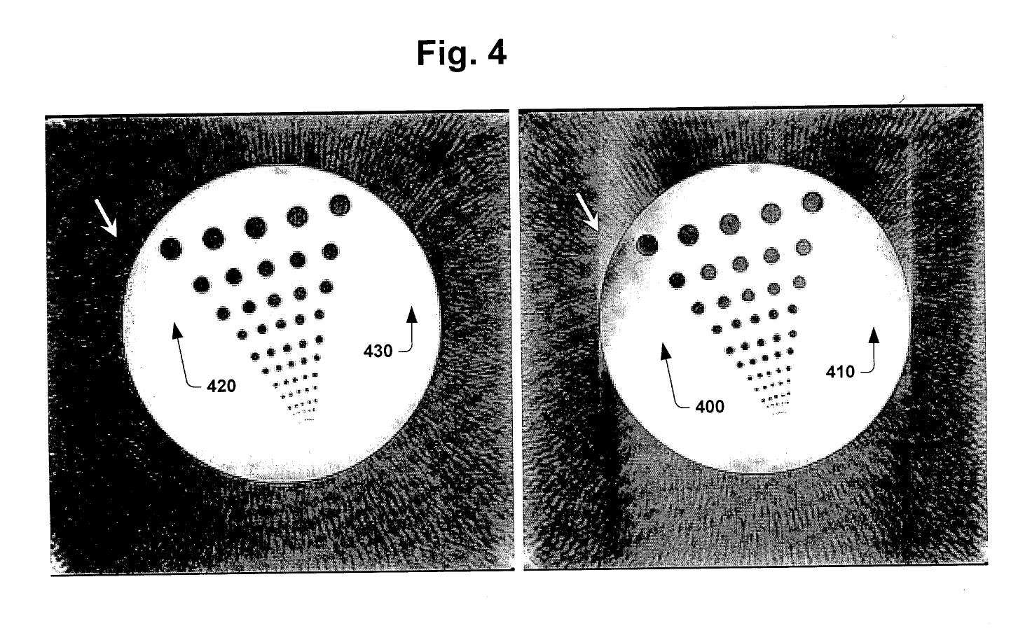

FIG. 3 illustrates an example k-space trajectory that does not exhibit errors due to timing and/or eddy currents. Note the unbroken concentric circles centered around the origin. From such an error free trajectory, it is more likely that images can be reconstructed using standard reconstruction techniques and not exhibit artifacts. FIG. 4 helps compare and contrast the images that can be reconstructed from data using planned, measured and/or predicted trajectories.

Thus, turning now to FIG. 4, the right hand image illustrates streak artifacts at locations 400 and 410. The right hand image was reconstructed using data associated with a planned trajectory. The left hand image illustrates mitigation of the streak artifacts at the locations 420 and 430 corresponding to the locations 400 and 410. The left hand image was reconstructed using data associated with an actual measured trajectory. Similar artifact mitigating effects can be achieved by employing one or more predicted trajectories in reconstruction. Various reconstruction techniques (e.g., B M Dale, et al. IEEE Trans. Med. Imag. 20(3): 207-217. 2001) can be employed to reconstruct an image from the MRI data.

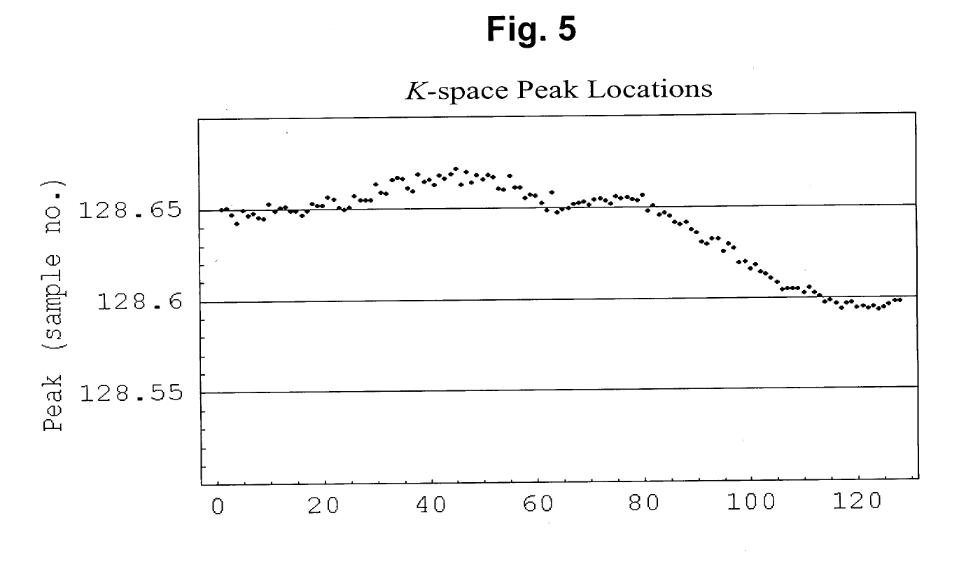

FIG. 5 illustrates a plot of k-space peak locations employed in predicting and/or measuring a trajectory. Determining and/or measuring a predicted trajectory can be facilitated by locating k-space peaks for one or more views. The k-space peaks can be located, for example, by sinc-interpolation of raw data. The sinc-interpolation can be done, for example, algebraically to mitigate peak-location discretization from FFT-based sinc-interpolation. Thus, a peak can be found by the conjugate-gradient method, for example. It is to be appreciated that other peak finding methods can be employed in accordance with the systems, methods, and so on described herein.

FIG. 5 illustrates one example plot of the location of actual k-space center locations obtained through sinc-interpolation of measured data. In the example, the average peak location within a radial view is at sample 128.641 with a range of 128.672-128.593=0.079. The example data was acquired symmetrically with TR/TE=10 ms/5 ms, 128 views and 256 samples/view.

A small total range for k-space peak locations indicates timing that is consistent between views even though such timing may not be consistent with a designed timing. Since such ranges of k-space peak locations can be anticipated, modeled, and/or predicted, artifact reduction can be achieved by reconstructing data with one or more predicted trajectories. When predicted trajectories are employed, the actual trajectory may or may not be measured. A predicted trajectory may, for example, assume a constant k-space offset for the views.

When one or more predicted trajectories are available to reconstruct data, entities including, but not limited to, a sequence programmer, a user, an artificial intelligence agent, a neural network, and so on could adjust the offset and/or select between various offsets to facilitate reducing artifact energy.

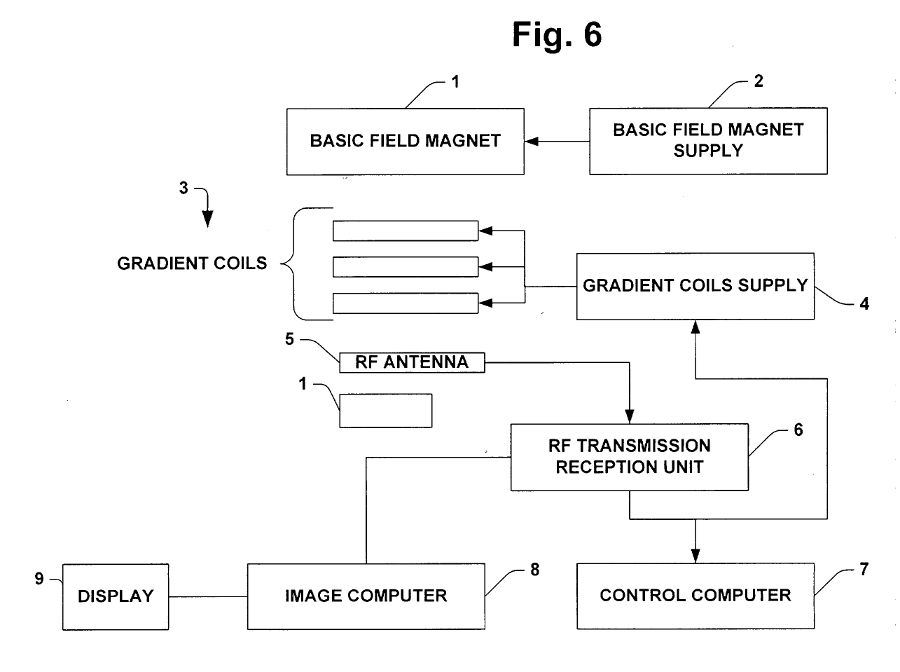

FIG. 6 illustrates one example magnetic resonance apparatus. Other MRI apparatus are known to those skilled in the art and along with other well-known systems are not illustrated herein for the sake of brevity. The apparatus includes a basic field magnet 1 supplied by a basic field magnet supply 2. The system has gradient coils 3 for respectively emitting gradient magnetic fields G_{S}, G_{P} and G_{R}, operated by a gradient coils supply 4. An RF antenna 5 is provided for generating the RF pulses, and for receiving the resulting magnetic resonance signals from an object being imaged. The RF antenna 5 is operated by an RF transmission/reception unit 6. The RF antenna 5 may be employed for transmitting and receiving, or alternatively, separate coils can be employed for transmitting and receiving. The gradient coils supply 4 and the RF transmission/reception unit 6 are operated by a control computer 7 to produce radio frequency pulses that are directed to the object to be imaged. The magnetic resonance signals received from the RF antenna 5 are subject to a transformation process, such as a two dimensional fast Fourier Transform (FFT), which generates pixelated image data. The transformation may be performed by an image computer 8 or other similar processing device. The image data may then be shown on a display 9. In one example, the MRI apparatus can acquire data according to a planned radial EPI sequence (e.g., Spider, rEPI, radial turbo SE).

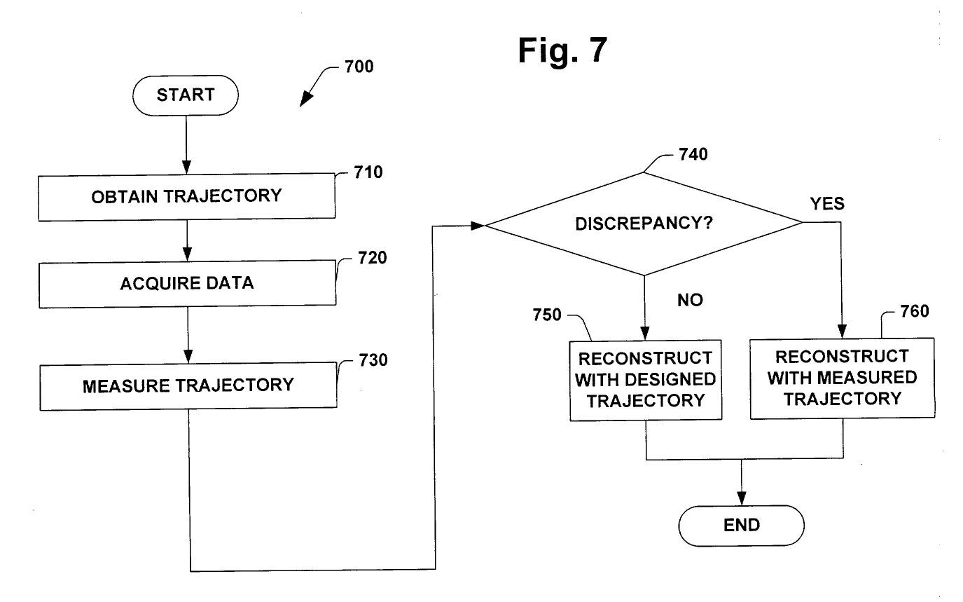

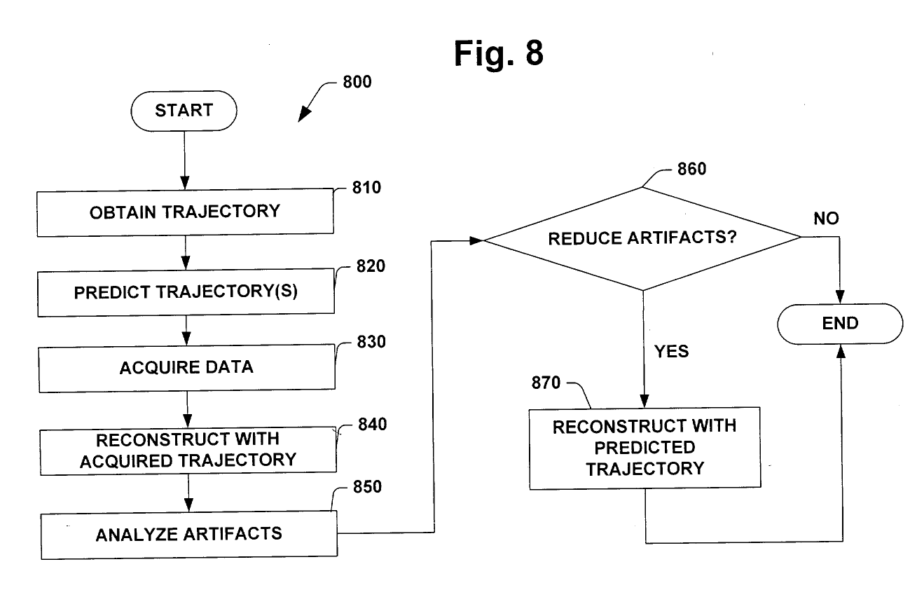

In view of the exemplary systems shown and described herein, example computer implemented methodologies will be better appreciated with reference to the flow diagrams of FIGS. 7 and 8. While for purposes of simplicity of explanation, the illustrated methodologies are shown and described as a series of blocks, it is to be appreciated that the methodologies are not limited by the order of the blocks, as some blocks can occur in different orders and/or concurrently with other blocks from that shown and described. Moreover, less than all the illustrated blocks may be required to implement an example methodology. Furthermore, additional and/or alternative methodologies can employ additional, not illustrated blocks. In one example, methodologies are implemented as computer executable instructions and/or operations stored on computer readable media including, but not limited to an application specific integrated circuit (ASIC), a compact disc (CD), a digital versatile disk (DVD), a random access memory (RAM), a read only memory (ROM), a programmable read only memory (PROM), an electronically erasable programmable read only memory (EEPROM), a disk, a carrier wave, and a memory stick. It is to be appreciated that the methodologies can be implemented in software as that term is defined herein.

In the flow diagrams, rectangular blocks denote “processing blocks” that may be implemented, for example, in software. Similarly, the diamond shaped blocks denote “decision blocks” or “flow control blocks” that may also be implemented, for example, in software. Alternatively, and/or additionally, the processing and decision blocks can be implemented in functionally equivalent circuits like a digital signal processor (DSP), an ASIC, and the like.

A flow diagram does not depict syntax for any particular programming language, methodology, or style (e.g., procedural, object-oriented). Rather, a flow diagram illustrates functional information one skilled in the art may employ to program software, design circuits, and so on. It is to be appreciated that in some examples, program elements like temporary variables, routine loops, and so on are not shown.

FIG. 7 is a flow chart of one example method 700 for reconstructing an image from MRI data, where the method employs a measured trajectory to facilitate mitigating problems associated with artifacts in a reconstructed image. At 710, a planned trajectory is obtained. For example, the planned trajectory may be acquired, accessed, loaded and/or generated. A trajectory can be accessed from locations including, but not limited to, a data base, a file, the Internet, a computer component, a local area network, a disk, a CD, a DVD, a RAM, a ROM, an ASIC, and so on. The planned trajectory may have been designed by entities including, but not limited to, the entity responsible for performing the image reconstruction, the MRI manufacturer, and a third party. The trajectory may be generated by, for example, a programmed computer component, a radiologist, a physician, a technician, an artificial intelligence program, a neural network, and so on.

At 720, k-space data is acquired. In one example, the data can be acquired according to a planned radial EPI sequence (e.g., Spider, rEPI, radial turbo SE). During and/or after 720, at 730, the actual trajectory experienced during the k-space data acquisition is measured. It is to be appreciated that the data can be acquired at 720 and that the actual trajectory can be measured in manners including, but not limited to, substantially in parallel and in serial.

At 740, a determination is made concerning the existence and/or type of discrepancy between the planned trajectory and the measured trajectory. If there is a discrepancy, and/or if the discrepancy is greater than a pre-determined, configurable threshold, then at 760 an image can be reconstructed from the data acquired during 720 with reference to the measured trajectory. In one example, the decision at 740 is omitted and images are reconstructed from measured trajectory data. Artifacts that might have appeared in the reconstructed image can thus be suppressed, removed, reduced, and/or minimized. If there is no discrepancy, and/or if the discrepancy is less than a pre-determined, configurable threshold, then at 750 an image can be reconstructed from the data acquired during 720 with reference to the planned trajectory and/or the measured trajectory. Thus, processing cycles can be more efficiently allocated to produce an improved reconstructed image than is conventional.

In one example, the image is selectively reconstructed from the data acquired during 720 with reference to selected portions of the planned trajectory and the measured trajectory. While one reconstruction technique (B M Dale, et al.) is referenced herein, it is to be appreciated that the methods described herein can be employed with other reconstruction techniques.

FIG. 8 is a flow chart of another example method 800 for reconstructing an image from MRI data, where the method employs a predicted trajectory to facilitate mitigating problems associated with artifacts in a reconstructed image. At 810, a planned trajectory is obtained. Obtaining a planned trajectory can include, but is not limited to, accessing a trajectory, loading a trajectory, acquiring a trajectory, generating a trajectory, and so on. A trajectory can be acquired from locations including, but not limited to, a data base, a file, the Internet, a computer component, a local area network, a disk, a CD, a DVD, a RAM, a ROM, an ASIC, and so on.

At 820, one or more trajectories are predicted. The predictions can be made based on data including, but not limited to, timing error history of an apparatus, eddy current history of an apparatus, timing error history of a software, eddy current history of a software, timing error history of a computer component, eddy current history of a computer component, k-space peak location data, and so on. The predicted trajectories can be stored, for example, in a data base or other similar computer component to facilitate retrieval and use at subsequent points in the method 800.

At 830, k-space data is acquired and at 840, an image can be reconstructed from the data acquired at 830. At 850, the reconstructed image is analyzed to detect the existence and/or nature of artifacts located in the image. It is to be appreciated that the analysis at 850 can be performed by a computer component and/or by a human examiner. At 860, a determination is made concerning whether to attempt to reduce artifacts in the reconstructed image. For example, the image may be so fatally flawed that attempts to remove artifacts are not warranted. Contrarily, the image may have no artifacts or an acceptable type/number/severity of artifacts so that artifact reduction is not undertaken. If the determination at 860 is NO, then processing can conclude.

But if the determination at 860 is YES, then at 870 the data acquired at 830 can be reconstructed into an image using one or more of the trajectories predicted at 820. For example, the analysis of 850 may reveal that the acquired data is consistent with a known time shift and that such time shift has been anticipated in a first predicted trajectory. Thus, data associated with the first predicted trajectory can be employed in reconstructing the image to facilitate removing artifacts associated with the time shift. Similarly, the analysis of 850 may reveal that the acquired data is consistent with a known eddy current and/or eddy current and time shift combination and that such current and/or current/shift combination has been anticipated in a second predicted trajectory. Thus, data associated with the second predicted trajectory can be employed in reconstructing the image to facilitate removing artifacts. While time shift and eddy currents are described above, it is to be appreciated that trajectories associated with other discrepancy introducing anomalies can be predicted and employed by the systems and methods described herein. For example, the image reconstructed at 870 can then be re-analyzed at 850 with subsequent decisions concerning further reducing artifacts. In another example, processing concludes after 870. In another example, predicting trajectories, acquiring data, analyzing the data and/or image and determining whether to reduce artifacts and/or repeat the prediction/acquisition cycle can occur.

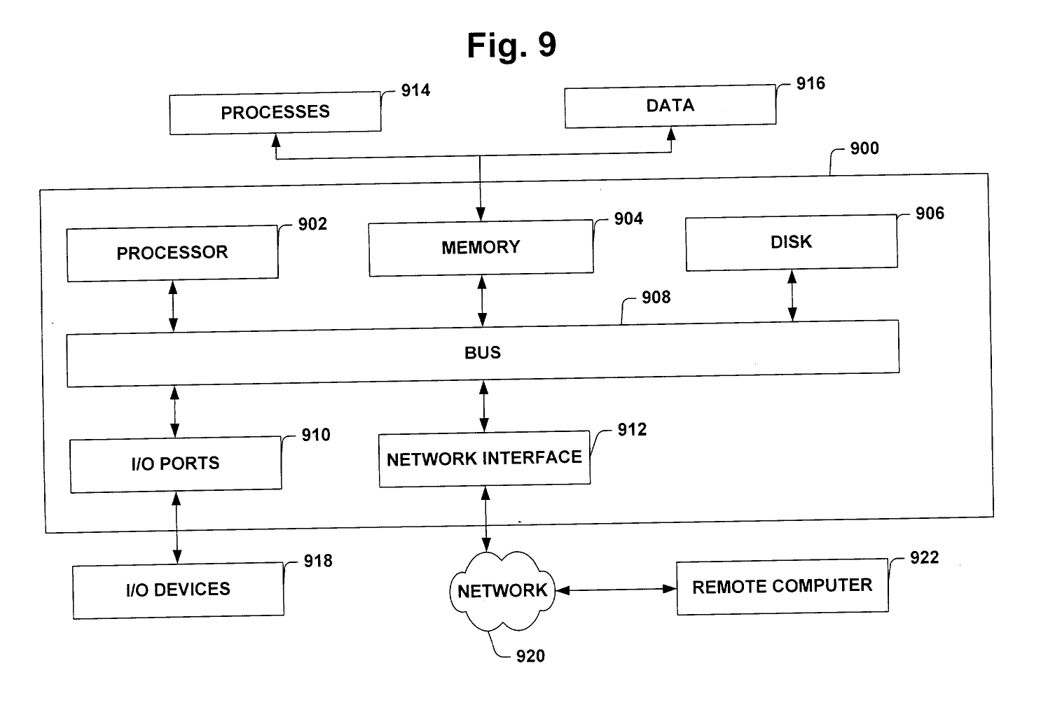

FIG. 9 illustrates a computer 900 that includes a processor 902, a memory 904, a disk 906, input/output ports 910, and a network interface 912 operably connected by a bus 908. Executable components of the systems described herein may be located on a computer like computer 900. Similarly, computer executable methods described herein may be performed on a computer like computer 900. It is to be appreciated that other computers may also be employed with the systems and methods described herein. Furthermore, it is to be appreciated that the computer 900 can be located locally to an MRI system or other radial data acquisition system, remotely to an MRI system or other radial data acquisition system, and/or can be embedded in an MRI or other radial data acquisition system.

The processor 902 can be of a variety of various processors including dual microprocessor and other multi-processor architectures. The memory 904 can include volatile memory and/or non-volatile memory. The non-volatile memory can include, but is not limited to, ROM, PROM, electrically programmable read only memory (EPROM), EEPROM, and the like. Volatile memory can include, for example, RAM, synchronous RAM (SRAM), dynamic RAM (DRAM), synchronous DRAM (SDRAM), double data rate SDRAM (DDR SDRAM), and direct RAM bus RAM (DRRAM). The disk 906 can include, but is not limited to, devices like a magnetic disk drive, a floppy disk drive, a tape drive, a Zip drive, a flash memory card, and/or a memory stick. Furthermore, the disk 906 can include optical drives like, a compact disk ROM (CD-ROM), a CD recordable drive (CD-R drive), a CD rewriteable drive (CD-RW drive) and/or a digital versatile ROM drive (DVD ROM). The memory 904 can store processes 914 and/or data 916, for example. The disk 906 and/or memory 904 can store an operating system that controls and allocates resources of the computer 900.

The bus 908 can be a single internal bus interconnect architecture and/or other bus architectures. The bus 908 can be of a variety of types including, but not limited to, a memory bus or memory controller, a peripheral bus or external bus, and/or a local bus. The local bus can be of varieties including, but not limited to, an industrial standard architecture (ISA) bus, a microchannel architecture (MSA) bus, an extended ISA (EISA) bus, a peripheral component interconnect (PCI) bus, a universal serial bus (USB), and a small computer systems interface (SCSI) bus.

The computer 900 interacts with input/output devices 918 via input/output ports 910. Input/output devices 918 can include, but are not limited to, a keyboard, a microphone, a pointing and selection device, cameras, video cards, displays, and the like. The input/output ports 910 can include but are not limited to, serial ports, parallel ports, and USB ports.

The computer 900 can operate in a network environment and thus is connected to a network 920 by a network interface 912. Through the network 920, the computer 900 may be logically connected to a remote computer 922. The network 920 includes, but is not limited to, local area networks (LAN), wide area networks (WAN), and other networks. The network interface 912 can connect to local area network technologies including, but not limited to, fiber distributed data interface (FDDI), copper distributed data interface (CDDI), ethernet/IEEE 802.3, token ring/IEEE 802.5, and the like. Similarly, the network interface 912 can connect to wide area network technologies including, but not limited to, point to point links, and circuit switching networks like integrated services digital networks (ISDN), packet switching networks, and digital subscriber lines (DSL).

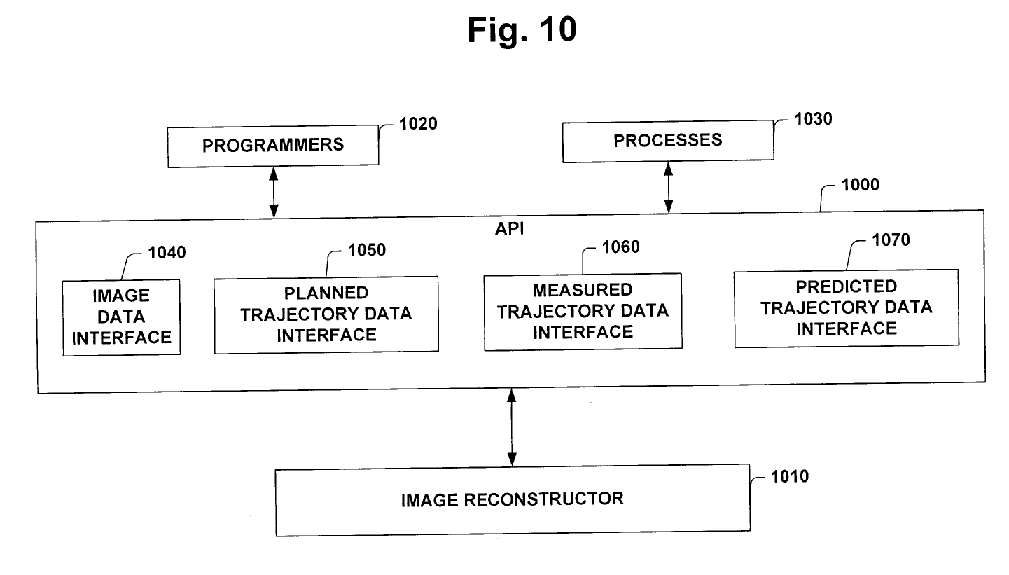

Referring now to FIG. 10, an application programming interface (API) 1000 is illustrated providing access to a system 1010 for reconstructing images. The API 1000 can be employed, for example, by programmers 1020 and/or processes 1030 to gain access to processing performed by the system 1010. For example, a programmer 1020 can write a program to access (e.g., to invoke its operation, to monitor its operation, to access its functionality) an image reconstructor 1010 where writing such a program is facilitated by the presence of the API l000. Rather than the programmer 1020 having to understand the internals of the image reconstructor 1010, the programmer's task is simplified by merely having to learn the interface 1000 to the image reconstructor 1010. This facilitates encapsulating the functionality of the image reconstructor 1010 while exposing that functionality. Similarly, the API 1000 can be employed to provide data values to the system 1010 and/or to retrieve data values from the system 1010. For example, a programmer 1020 may wish to present image data to the image reconstructor 1010 and thus the programmer 1020 may employ an image data interface 1040 component of the API 1000. Similarly, the programmer 1020 may wish to present planned trajectory data to the image reconstructor 1010 and thus may employ a planned trajectory data interface 1050 component of the API 1000. After receiving the image data and planned trajectory data, the image reconstructor 1010 may, for example, pass a reconstructed image to a process 1030. Similarly, a process 1030 may desire to present measured trajectory data to the image reconstructor and thus may employ the measured trajectory data interface 1060 component of the API 1000. Furthermore, a process 1030 may desire to present predicted trajectory data to the image reconstructor 1010 and thus may employ the predicted trajectory data interface 1070 component of the API 1000 to effect such transfer.

Thus, in one example of the API 1000, a set of application program interfaces can be stored on a computer-readable medium. The interfaces can be executed by a computer component to gain access to an image reconstructor. Interfaces can include, but are not limited to, a first interface that receives and/or returns an image data associated with an image, a second interface that receives and/or returns a trajectory data associated with a planned trajectory, a third interface that receives and/or returns a data associated with a measured trajectory data, and a fourth interface that receives and/or returns a data associated with a predicted trajectory data where the interfaces facilitate interacting with an image reconstructor that mitigates problems associated with artifacts in images reconstructed from k-space data.

The systems, methods, and objects described herein may be stored, for example, on a computer readable media. Media can include, but are not limited to, an ASIC, a CD, a DVD, a RAM, a ROM, a PROM, a disk, a carrier wave, a memory stick, and the like. Thus, an example computer readable medium can store computer executable instructions for a computer implemented method for reconstructing an MRI data into an image so that artifacts in the image are reduced. An example method may include obtaining a planned radial trajectory, acquiring an MRI data, where it is attempted to acquire the MRI data in accordance with the planned trajectory, measuring a trajectory that occurs during data acquisition, and reconstructing the MRI data into an image using the measured trajectory.

In another example, a computer readable medium can store computer executable instructions for a computer implemented method for reconstructing an image from an MRI data that mitigates artifacts in the reconstructed image. An example method may include, obtaining a planned radial trajectory, predicting trajectories that may occur during data acquisition, acquiring an MRI data, where it is attempted to acquire the MRI data in accordance with the planned trajectory, and selectively reconstructing the MRI data into an image using the predicted trajectories.

In another example, a computer readable medium can store computer executable instructions for a computer implemented method for reconstructing an image from an MRI data. The example method can include obtaining a planned radial trajectory, predicting trajectories, acquiring an MRI data, where it is attempted to acquire the data in accordance with the planned radial trajectory, and measuring a trajectory during data acquisition. The method also includes reconstructing an image from the MRI data in association with the planned radial trajectory then either analyzing the reconstructed image to determine an artifact level and/or determining a discrepancy level between the planned radial trajectory and the measured trajectory. Based on the analysis, the method includes selectively reconstructing the MRI data into an image using the planned radial trajectory, the measured trajectory, and/or the predicted trajectories based, at least in part, on the artifact level and/or the discrepancy level.

In yet another example, a computer readable medium can store computer executable instructions for a computer implemented method that reduces artifacts in reconstructed MRI data. An example method can include obtaining a planned radial trajectory, predicting trajectories that may occur during data acquisition, and acquiring an MRI data, where it is attempted to acquire the MRI data in accordance with the planned radial trajectory. The method can also include reconstructing an image from the MRI data by using the planned radial trajectory and then analyzing the reconstructed image to determine an artifact level. After reconstruction, the method can include selectively reconstructing the MRI data into an image using the planned radial trajectory and/or the predicted trajectories based, at least in part, on the artifact level.

In still another example, a computer readable medium can store computer executable instructions for a computer implemented method that reduces artifacts in reconstructed MRI data. The method can include obtaining a planned radial trajectory, predicting trajectories that may occur during data acquisition, acquiring an MRI data, where it is attempted to acquire the MRI data in accordance with the planned trajectory, and measuring a trajectory that occurs during data acquisition. After and/or substantially in parallel with data acquisition, the method includes reconstructing an image from the MRI data in association with the planned radial trajectory. After reconstruction, the method can selectively analyze the reconstructed image to determine an artifact level and/or determine a discrepancy level between the planned radial trajectory and the measured trajectory. Based on the artifact level and/or discrepancy level, the method includes selectively reconstructing the MRI data into an image using the planned radial trajectory, the measured trajectory, the actual trajectory, and/or the predicted trajectories.

Similarly, a computer readable medium can store computer executable components of a system for reconstructing an image from MRI data that mitigates artifacts in the image. An example system can include an MRI data, a planned trajectory data, an acquired trajectory data, and a data store for storing the MRI data, the planned trajectory data, and the acquired trajectory data. The system can also include a trajectory comparator for comparing the planned trajectory data with the acquired trajectory data to produce a trajectory comparison data. The system can also include an image reconstructor for reconstructing an image from the MRI data and the planned trajectory data and/or the acquired trajectory data based, at least in part, on the trajectory comparison data.

In another example, a computer readable medium can store computer executable components of a system for producing an MRI image. The system can include a magnetic resonance imager for acquiring an MRI data and an image reconstructor for reconstructing an image from the MRI data, where the image reconstructor employs one or more of, a planned trajectory, a measured trajectory, and a predicted trajectory to reconstruct the image.

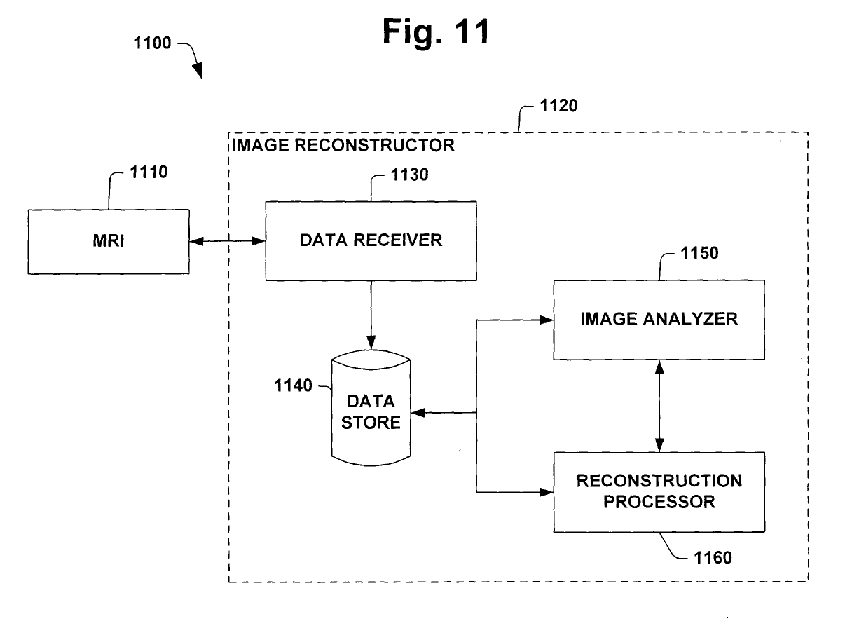

FIG. 11 illustrates an example system 1100 in which an MRI system 1110 interacts with an image reconstructor 1120. The image reconstructor 1120 can include, for example, a data receiver 1130, a data store 1140, an image analyzer 1150, and a reconstruction processor 1160. The MRI system 1110 can be operably connected to the image reconstructor 1120 by techniques including, but not limited to, direct connections, local area networks, wide area networks, satellite communications, cellular communications, and so on. The MRI 1110 provides data to the image reconstructor 1120 which then reconstructs an MRI image where the effects of artifacts produced from k-space data affected by, for example, timing delays and/or uncompensated eddy currents are mitigated. The effects can be mitigated by, for example, reconstructing an image by employing a measured trajectory and/or a predicted trajectory associated with the acquired k-space data.

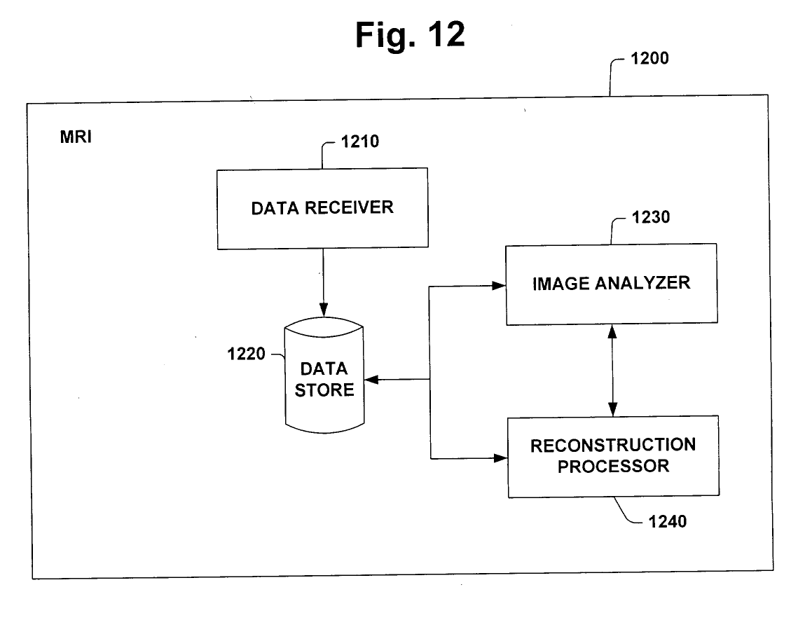

FIG. 12 illustrates an example MRI system 1200 that includes computer components that form an image reconstructor. The components of the image reconstructor include, but are not limited to, a data receiver 1210, a data store 1220, an image analyzer 1230, and a reconstruction processor 1240 that are substantially similar to the computer components described above in connection with FIG. 11. While the MRI 1110 (FIG. 11) was separate from the data receiver 1130 (FIG. 11), data store 1140 (FIG. 11), image analyzer 1150 (FIG. 11), and reconstruction processor 1160 (FIG. 11), in the system 1200, the image reconstructing components are incorporated into the MRI system. While FIGS. 11 and 12 show two example systems, one with separate components and one with incorporated components, it is to be appreciated that other examples may have a mixture of separate and incorporated components.

What has been described above includes several examples. It is, of course, not possible to describe every conceivable combination of components or methodologies for purposes of describing the methods, systems, computer readable media and so on employed in employing measured and/or predicted trajectories to improve images reconstructed from k-space data. However, one of ordinary skill in the art may recognize that further combinations and permutations are possible. Accordingly, this application is intended to embrace alterations, modifications, and variations that fall within the scope of the appended claims. Furthermore, to the extent that the term “includes” is employed in the detailed description or the claims, it is intended to be inclusive in a manner similar to the term “comprising” as that term is interpreted when employed as a transitional word in a claim.

What is claimed is:

1. A computer implemented method for reconstructing an image from an MRI data, comprising:
obtaining a planned radial trajectory;
acquiring an MRI data, where it is attempted to acquire the MRI data in accordance with the planned radial trajectory;
measuring a measured trajectory experienced while acquiring the MRI data; and
reconstructing the MRI data into an image using the measured trajectory.

2. The method of claim 1, where obtaining a planned radial trajectory comprises one or more of, accessing, acquiring, loading, and generating a planned radial trajectory.

3. A computer readable medium storing computer executable instructions operable to perform the method of claim 1.

4. A computer implemented method for reconstructing an image from an MRI data, comprising:
obtaining a planned radial trajectory;
acquiring an MRI data, where it is attempted to acquire the MRI data in accordance with the planned radial trajectory;
measuring a measured trajectory experienced while acquiring the MRI data;
determining a discrepancy level between the planned radial trajectory and the measured trajectory; and
selectively reconstructing the MRI data into an image using one of, the planned radial trajectory, and the measured trajectory based, at least in part, on the discrepancy level.

5. The method of claim 4, where obtaining a planned radial trajectory comprises one or more of, accessing, acquiring, loading, and generating a planned radial trajectory.

6. A computer readable medium storing computer executable instructions operable to perform the method of claim 4.

7. A computer implemented method for reconstructing an image from an MRI data, comprising:
obtaining a planned radial trajectory;
predicting one or more predicted trajectories;
acquiring an MRI data, where it is attempted to acquire the MRI data in accordance with the planned radial trajectory; and
selectively reconstructing the MRI data into an image using one or more of the predicted trajectories.

8. The method of claim 7, where obtaining a planned radial trajectory comprises one or more of, accessing, acquiring, loading, and generating a planned radial trajectory.

9. A computer readable medium storing computer executable instructions operable to perform the method of claim 7.

10. A computer implemented method for reconstructing an image from an MRI data, comprising:
obtaining a planned radial trajectory;
predicting one or more predicted trajectories;
acquiring an MRI data, where it is attempted to acquire the MRI data in accordance with the planned radial trajectory;
reconstructing an image from the MRI data in association with the planned radial trajectory;
analyzing the reconstructed image to determine an artifact level; and
selectively reconstructing the MRI data into an image using one or more of, the planned radial trajectory, and one or more of the predicted trajectories based, at least in part, on the artifact level.

11. The method of claim 10, where obtaining a planned radial trajectory comprises one or more of, accessing, acquiring, loading, and generating a planned radial trajectory.

12. A computer readable medium storing computer executable instructions operable to perform the method of claim 10.

13. A computer implemented method for reconstructing an image from an MRI data, comprising:
obtaining a planned radial trajectory;
predicting one or more predicted trajectories;
acquiring an MRI data, where it is attempted to acquire the MRI data in accordance with the planned radial trajectory;
measuring a measured trajectory;
reconstructing an image from the MRI data in association with the planned radial trajectory;
performing one or more of, analyzing the reconstructed image to determine an artifact level, and determining a discrepancy level between the planned radial trajectory and the measured trajectory; and
selectively reconstructing the MRI data into an image using one or more of, the planned radial trajectory, the measured trajectory, and the predicted trajectories based, at least in part, on one or more of, the artifact level, and the discrepancy level.

14. The method of claim 13, where obtaining a planned radial trajectory comprises one or more of, accessing, acquiring, loading, and generating a planned radial trajectory.

15. A computer readable medium storing computer executable instructions operable to perform the method of claim 13.

16. A system for reconstructing an image from an MRI data, comprising:
an MRI data;
a planned trajectory data;
an acquired trajectory data;
a data store for storing the MRI data, the planned trajectory data, and the acquired trajectory data;
a trajectory comparator for comparing the planned trajectory data with the acquired trajectory data to produce a first trajectory comparison data; and
an image reconstructor for reconstructing an image from the MRI data and one or more of, the planned trajectory data, and the acquired trajectory data based, at least in part, on the first trajectory comparison data.

17. The system of claim 16, comprising:
a predicted trajectory data;
where the trajectory comparator compares the predicted trajectory data with the acquired trajectory data to produce a second trajectory comparison data; and
where the image reconstructor reconstructs an image from the MRI data and one or more of, the planned trajectory data, the acquired trajectory data, and the predicted trajectory data based, at least in part, on one or more of, the first trajectory comparison data, and the second trajectory comparison data.

18. A computer readable medium storing computer executable components of the system of claim 16.

19. A computer readable medium storing computer executable components of the system of claim 17.

20. A set of application programming interfaces embodied on a computer readable medium for execution by a computer component in conjunction with an application program that reconstructs an image from MRI data, comprising:
a first interface for passing an image data between two or more of, a programmer, a process, and an image reconstructor;
a second interface for passing a planned trajectory data between two or more of, a programmer, a process, and an image reconstructor; and
a third interface for passing a measured trajectory data between two or more of, a programmer, a process, and an image reconstructor;
where the image reconstructor reconstructs an image from an MRI data from one or more of, an image data, a planned trajectory data, and a measured trajectory data.

21. The set of application programming interfaces of claim 20, comprising:
a fourth interface for passing a predicted trajectory data between two or more of, a programmer, a process, and an image reconstructor;
where the image reconstructor reconstructs an image from an MRI data from one or more of, an image data, a planned trajectory data, a measured trajectory data, and a predicted trajectory data.

22. A system for reconstructing a k-space data into an image, comprising:
means for receiving a k-space data;
means for accessing a planned radial trajectory data;
means for accessing a measured trajectory data;
means for producing a comparison of the planned radial trajectory data and the measured trajectory data; and
means for selectively reconstructing an image from the k-space data in a manner that mitigates artifacts in the image by selectively employing the planned radial trajectory data and the measured trajectory data when reconstructing the image based, at least in part, on the comparison between the planned radial trajectory data and the measured trajectory data.

23. The system of claim 22, where the k-space data is acquired by an MRI system.

24. A system for reconstructing a k-space data into an image, comprising:
means for receiving a k-space data;
means for accessing a planned radial trajectory data;
means for accessing a predicted trajectory data;
means for producing a comparison of the planned radial trajectory data and the predicted trajectory data; and
means for selectively reconstructing an image from the k-space data in a manner that mitigates artifacts in the image by selectively employing the planned radial trajectory data and the predicted trajectory data when reconstructing the image based, at least in part, on the comparison between the planned radial trajectory data and the predicted trajectory data.

25. A system for producing an MRI image, comprising:
a magnetic resonance imager for acquiring an MRI data; and
an image reconstructor for reconstructing an image from the MRI data, where the image reconstructor employs one or more of a measured trajectory, and a predicted trajectory to reconstruct the image.

26. The system of claim 25, the magnetic resonance imager comprising:
a polarizing magnetic field generator for generating a polarizing magnetic field in an examination region;
an RF generator for generating an excitation magnetic field that produces transverse magnetization in nuclei subjected to the polarizing magnetic field;
a sensor for sensing a magnetic resonance signal produced by the transverse magnetization;
a gradient generator for generating a magnetic field gradient to impart a read component into the magnetic resonance signal, where the read component indicates a location of a transversely magnetized nuclei along a first projection axis, the gradient generator generating subsequent magnetic field gradients to impart subsequent read components into the magnetic resonance signal that indicates subsequent locations of the transversely magnetized nuclei along subsequent projection axes;
a pulse controller operably coupled to the RF generator, the gradient generator, and the sensor, the pulse controller conducting a scan in which a series of data points are acquired at read points along a radial axis to form a magnetic resonance data view, subsequent magnetic resonance data views defining a magnetic resonance data set;
a data store for storing the magnetic resonance data set; and
a processor for reconstructing an image array for a display from the stored magnetic resonance data set.

27. The system of claim 26 where the image reconstructor is physically located inside the magnetic resonance imager.

28. The system of claim 26 where the image reconstructor is physically separate from the magnetic resonance imager.

29. The system of claim 25, the image reconstructor comprising:
a data receiver for receiving an MRI data from the magnetic resonance imager;
a data store for storing one or more of an MRI data, a planned trajectory data, a measured trajectory data, a predicted trajectory data, and a reconstructed image;
an image analyzer for one or more of analyzing a reconstructed image for an artifact and analyzing a measured trajectory data to determine whether a measured trajectory varied from a planned trajectory; and
a reconstruction processor for reconstructing an image from an MRI data and one or more of, a planned trajectory, a measured trajectory, and a predicted trajectory.

30. A system for producing an image from a k-space data, comprising:
a k-space data acquirer for acquiring a k-space data; and
an image reconstructor for reconstructing an image from the k-space data, where the image reconstructor employs one or more of, a measured trajectory, and a predicted trajectory to reconstruct the image.

31. The system of claim 30, where the k-space data is acquired from an MRI system.
